SEQUENCE LISTING (9 sequences)

SEQ ID NO: 1 - protein, 375 aa, Lactobacillus casei
ATGAAAAAAAAGATTATCTCAGCTATTTTAATGTCTACAGTGATACTTTCTGCTGCAGCCCCGTTGTCAGGTGTTTATGCATCAGCAGCTGTCACGGGAGTTCCTGTTAAAGGTCAAGACACTGTCAAAGGCCGTGTTCCATTCAATGGACAAGATCCCGTTAAAGGACAAGTTTCAGTTAAAGGTCAAGATAAAGTCAAAGCGCAAGAGCCAGTCAAAGGTCCAGTCTCCACTAAGCCTGGCTCCTGCCCCATTATCTTGATCCGGTGCGCCATGTTGAATCCCCCTAACCGCTGCTTGAAAGATACTGACTGCCCAGGAATCAAGAAGTGCTGTGAAGGCTCTTGCGGGATGGCCTGTTTCGTTCCCCAGTGA

SEQ ID NO: 2 - protein, 3874 aa, Lactobacillus casei
ATGAACTTTAATAAAATTGATTTAGACAATTGGAAGAGAAAAGAGATATTTAATCATTATTTGAACCAACAAACGACTTTTAGTATAACCACAGAAATTGATATTAGTGTTTTATACCGAAACATAAAACAAGAAGGATATAAATTTTACCCTGCATTTATTTTCTTAGTGACAAGGGTGATAAACTCAAATACAGCTTTTAGAACTGGTTACAATAGCGACGGAGAGTTAGGTTATTGGGATAAGTTAGAGCCACTTTATACAATTTTTGATGGTGTATCTAAAACATTCTCTGGTATTTGGACTCCTGTAAAGAATGACTTCAAAGAGTTTTATGATTTATACCTTTCTGATGTAGAGAAATATAATGGTTCGGGGAAATTGTTTCCCAAAACACCTATACCTGAAAATGCTTTTTCTCTTTCTATTATTCCATGGACTTCATTTACTGGGTTTAACTTAAATATCAATAATAATAGTAATTACCTTCTACCCATTATTACAGCAGGAAAATTCATTAATAAAGGTAATTCAATATATTTACCGCTATCTTTACAGGTACATCATTCTGTTTGTGATGGTTATCATGCAGGATTGTTTATGAACTCTATTCAGGAATTGTCAGATAGGCCTAATGACTGGCTTTTATAATATGAGATAATGCCGACTGTACTTTCGGATCCTAAACGCAATTGATGATTGGTTCGGAAGGCACGTTAGGAATCATTACCGAAGTAATCGTTAAACTGTTGCCGATTCCGCTAGGGACCCATAACTTCGTATAATGTATGCTATACGAACGGTACAGCCCGGGCATGAGCTCCGATCGCTACGAGAAGACGCACTATCGACCATACCTAATAATTTATCTACATTCCCTTTAGTAACGTGAAGAAGATCTCTAAAGCTGACGGGGTAAACTATATAAAATCCAAATAAATTTCTAAAAATAAAAAAGTCTGTCGATGAACAGACTTTTTTATTATAGTTTAAAGCAAACTTTTAAATATAATAAAAAGAGTTAGTTGAAATTTTCTACTAACTCTTTTTTATTTTTAGTTTTTAACTGCAGAAGCAAATTCTTCTTTAGCAAAAGCTTCATCGATGATAGCTTTCAATTGAGCGTGTAACTTTCCAAATTTACAAAAGCGACTCATAGAATTATTTCCTCCCGTTAAATAATAGATAACTATTAAAAATAGACAATACTTGCTCATAAGTAACGGTACTTAAATTGTTTACTTTGGCGTGTTTCATTGCTTGATGAAACTGATTTTTAGTAAACAGTTGACGATATTCTCGATTGACCCATTTTGAAACAAAGTACGTATATAGCTTCCAATATTTATCTGGAACATCTGTGGTATGGCGGGTAAGTTTTATTAAGACACTGTTTACTTTTGGTTTAGGATGAAAGCATTCCGCTGGCAGCTTAAGCAATTGCTGAATCGAGACTTGAGTGTGCAAGAGCAACCCTAGTGTTCGGTGAATATCCAAGGTACGCTTGTAGAATCCTTCTTCAACAATCAGATAGATGTCAGACGCATGGCTTTCAAAAACCACTTTTTTAATAATTTGTGTGCTTAAATGGTAAGGAATACTCCCAACAATTTTATACCTCTGTTTGTTAGGGAATTGAAACTGTAGAATATCTTGGTGAATTAAAGTGACACGAGTATTCAGTTTTAATTTTTCTGACGATAAGTTGAATAGATGACTGTCTAATTCAATAGACGTTACCTGTTTACTTATTTTAGCCAGTTTCGTCGTTAAATGCCCTTTACCTGTTCCAATTTCGTAAACGGTATCGGTTTCTTTTAAATTCAATTGTTTTATTATTTGGTTGAGTACTTTTTCACTCGTTAAAAAGTTTTGAGAATATTTTATATTTTTGTTCATGTAATCACTCCTTCTTAATTACAAATTTTTAGCATCTAATTTAACTTCAATTCCTATTATACAAAATTTTAAGATACTGCACTATCAACACACTCTTAAGTTTGCTTCTAAGTCTTATTTCCATAACTTCTTTTACGTTTCCGCCATTCTTTGCTGTTTCGATTTTTATGATATGGTGCAAGTCAGCACGAACACGAACCGTCTTATCTCCCATTATATCTTTTTTTGCACTGATTGGTGTATCATTTCGTTTTTCTTTTGCGGACCTGCAGATGCGATATCATGCGCATGCAAGCTTATCGATGATAAGCTGTCAAACATGAGAATTACAACTTATATCGTATGGGGCTGACTTCAGGTGCTACATTTGAAGAGATAAATTGCACTGAAATCTAGAAATATTTTATCTGATTAATAAGATGATCTTCTTGAGATCGTTTTGGTCTGCGCGTAATCTCTTGCTCTGAAAACGAAAAAACCGCCTTGCAGGGCGGTTTTTCGAAGGTTCTCTGAGCTACCAACTCTTTGAACCGAGGTAACTGGCTTGGAGGAGCGCAGTCACCAAAACTTGTCCTTTCAGTTTAGCCTTAACCGGCGCATGACTTCAAGACTAACTCCTCTAAATCAATTACCAGTGGCTGCTGCCAGTGGTGCTTTTGCATGTCTTTCCGGGTTGGACTCAAGACGATAGTTACCGGATAAGGCGCAGCGGTCGGACTGAACGGGGGGTTCGTGCATACAGTCCAGCTTGGAGCGAACTGCCTACCCGGAACTGAGTGTCAGGCGTGGAATGAGACAAACGCGGCCATAACAGCGGAATGACACCGGTAAACCGAAAGGCAGGAACAGGAGAGCGCACGAGGGAGCCGCCAGGGGGAAACGCCTGGTATCTTTATAGTCCTGTCGGGTTTCGCCACCACTGATTTGAGCGTCAGATTTCGTGATGCTTGTCAGGGGGGCGGAGCCTATGGAAAAACGGCTTTGCCGCGGCCCTCTCACTTCCCTGTTAAGTATCTTCCTGGCATCTTCCAGGAAATCTCCGCCCCGTTCGTAAGCCATTTCCGCTCGCCGCAGTCGAACGACCGAGCGTAGCGAGTCAGTGAGCGAGGAAGCGGAATATATCCTGTATCACATATTCTGCTGACGCACCGGTGCAGCCTTTTTTCTCCTGCCACATGAAGCACTTCACTGACACCCTCATCAGTGCCAACATAGTAAGCCAGTATACACTCCGCTAGCGCTGATGTCCGGCGGTGCTTTTGCCGTTACGCACCACCCCGTCAGTAGCTGAACAGGAGGGACAGCGTGTTGCTTTGATTGATAGCCAAAAAGCAGCAGTTGATAAAGCAATTACTGATATTGCTGAAAAATTGTAATTTATAAATAAAAATCACCTTTTAGAGGTGGTTTTTTTATTTATAAATTATTCGTTTGATTTCGCTTTCGATAGAACAATCAAAGCGAGAATAAGGAAGATAAATCCCATAAGGGCGGGAGCAGAATGTCCGAGACTAATGTAAATTTGTCCACCAATTAAAGGACCGATAACGCGCTCGAGCCTGATAGAAACAGAAGCCACTGGAGCACGTTTAAACAATTTAAATCTACCGTTCGTATAATGTATGCTATACGAAGTTATGACAATGTCTTAGGCGTTAAGGTCGTTTTAGCCGATGGTCGCGAAGTTAAGTAAGGTACCATGCAGTTTAAATTCGGTCCTCGGGATATGATAAGATTAATAGTTTTAGCTATTAATCTTTTTTTATTTTTATTTAAGAATGGCTTAATAAAGCGGTTACTTTGGATTTTTGTGAGCTTGGACTAGAAAAAAACTTCACAAAATGCTATACTAGGTAGGTAAAAAAATATTCGGAGGAATTTTGAAATGGCAATCGTTTCAGCAGAATGCAGATGAAGAAAGCAGACAAGTAAGCCTCCTAAATTCACTTTAGATAAAAATTTAGGAGGCATATCA

SEQ ID NO: 3 - protein, 6668 aa, Lactobacillus casei
ATGAACTTTAATAAAATTGATTTAGACAATTGGAAGAGAAAAGAGATATTTAATCATTATTTGAACCAACAAACGACTTTTAGTATAACCACAGAAATTGATATTAGTGTTTTATACCGAAACATAAAACAAGAAGGATATAAATTTTACCCTGCATTTATTTTCTTAGTGACAAGGGTGATAAACTCAAATACAGCTTTTAGAACTGGTTACAATAGCGACGGAGAGTTAGGTTATTGGGATAAGTTAGAGCCACTTTATACAATTTTTGATGGTGTATCTAAAACATTCTCTGGTATTTGGACTCCTGTAAAGAATGACTTCAAAGAGTTTTATGATTTATACCTTTCTGATGTAGAGAAATATAATGGTTCGGGGAAATTGTTTCCCAAAACACCTATACCTGAAAATGCTTTTTCTCTTTCTATTATTCCATGGACTTCATTTACTGGGTTTAACTTAAATATCAATAATAATAGTAATTACCTTCTACCCATTATTACAGCAGGAAAATTCATTAATAAAGGTAATTCAATATATTTACCGCTATCTTTACAGGTACATCATTCTGTTTGTGATGGTTATCATGCAGGATTGTTTATGAACTCTATTCAGGAATTGTCAGATAGGCCTAATGACTGGCTTTTATAATATGAGATAATGCCGACTGTACTTTCGGATCCTAAACGCAATTGATGATTGGTTCGGAAGGCACGTTAGGAATCATTACCGAAGTAATCGTTAAACTGTTGCCGATTCCGCTAGGGACCCATAACTTCGTATAATGTATGCTATACGAACGGTACAGCCCGGGCATGAGCTCGTTTAAGCTTAGTCTTATAACTATACTGACAATAGAAACATTAACAAATCTAAAACAGTCTTAATTCTATCTTGAGAAAGTATTGGTAATAATATTATTGTCGATAACGCGAGCATAATAAACGGCTCTGATTAAATTCTGAAGTTTGTTAGATACAATGATTTCGTTCGAAGGAACTACAAAATAAATTATAAGGAGGCACTCAAAATGAGTACAAAAGATTTTAACTTGGATTTGGTATCTGTTTCGAAGAAAGATTCAGGTGCATCACCACGCATTACAAGTATTTCGCTATGTACACCCGGTTGTAAAACAGGAGACTCTGCATGGATCCCCCGTCTGAACGAACTTAATGGGAGGAAAAATTAAAAAAGAACAGTTATGAAAAAAAAGATTATCTCAGCTATTTTAATGTCTACAGTGATACTTTCTGCTGCAGCCCCGTTGTCAGGTGTTTATGCATCAGCAGCTGTCACGGGAGTTCCTGTTAAAGGTCAAGACACTGTCAAAGGCCGTGTTCCATTCAATGGACAAGATCCCGTTAAAGGACAAGTTTCAGTTAAAGGTCAAGATAAAGTCAAAGCGCAAGAGCCAGTCAAAGGTCCAGTCTCCACTAAGCCTGGCTCCTGCCCCATTATCTTGATCCGGTGCGCCATGTTGAATCCCCCTAACCGCTGCTTGAAAGATACTGACTGCCCAGGAATCAAGAAGTGCTGTGAAGGCTCTTGCGGGATGGCCTGTTTCGTTCCCCAGTGAGGACTAGTGAATTCGCGGCCGCCTGCAGGTCGACGGTATCGATAGCCCGCCTAATGAGCGGGCTTTTTTTTGATATCAAGCTTATCGATACCGTCGACCTCGAGTGCATATTTTCGGCAATCTTCTCAATGAGATGCTCTTCAGCATGTTCAATGATGTCGATTTTTTATTAAAACGTCTCAAAATCGTTTCTGAAAACGAAGCACATGCTTGGGCTGCCTTGTCGGCGAGAAAGCTTATTATTGGCGATATCGAAGCTGTTTAAGTGACGGTTTTGACTGATTGCAGTACCATCAGACGTATCAAAAACGAGGGGGATTTTAAATGGTAGCATTTTTGTGGGCGCAGGATCGGGATGGTGTAATCGGTAAAGACGGCCATTTGCCATGGCATTTGCCAGATGATTTGCATTATTTCCGGACTCAGACTGAAGGAAAAATGATGGTGGTTGGGCGTCGCACGTACGAAAGTTTTCCAAAACGGCCATTACCAGATCGTACGAACGTGGTTTTGACGCACCAGGCTGATTACCAGGCACCAGGCGCGATTGTCTTGCATCAGGTTGCTGAAGTGCTTGATTATGCGAAGGAACATGCAGATCAGGCATTAGTCATCGCCGGTGGTGCTCAAATCTTTAGCGCCTTTAAAGACATGGTTGATACCTTGCTCGTGACCCGTCTAGCTGGCAGTTTTGCAGGTGACACTAAAATGATTCCACTAGATTGGGATGCGTTTACTAAAACCTCAAGCCGAACTGTCGAAGATCAAAACCCCGCTTTGACGCATACTTATGAAGTTTGGCAAAAGCAAAAATGATCTGACGCGTTTAGCAGCTAAAGAAATTTTTTAAGGAACTTAAATAGTTATGTGCATTTGTAGTTCGTTTTTTTAACTAAAATTGACTCATGTGCAAAAAAGATCGGCTTCTCCGTCTGACGGAGCGAGCCGATCTTTTTTATATAGTTAGCATTAAACGAAAAGGTAAATTGAAATGTACATGCACAGGCTGCCGAGAATGACAAACAGGTGCCAGATGACATGGATGTAGGGGATGCCTTTTTGCAGGTACAGCATAGCGCCACCTGTGTATGCTAACCCGCCAGCAACCAGTAACCAGAAGCCAATCGGTCCTAAGTGATGCCACAGTGGCACCATGCCGATTAAGCAAAGCCAGCCGAGAATGACGTAAATCATGGTTTCCAGATGCTTGAAGCGATTCAAGAAGAACAGCTTGTAGAGGATGCCGCCAAAGCAAAGCGCCCAGATGGCAATTAGCAAGCCGATCCCTAATGGTCCGCCAATCGCGACCAAACAGTAAGGCAGGTAGGAGCCAGCGATTAATATGAAAACGCCAGAGTGATCTAGGACCTGTAGGACATGGCGCGCCTTGCTAAAATAGAAACCGTGGAACGCCGTTGAAGCCGTATATAAGATGATGAGGGAAATCCCAAATCCTAAGTAACTGATTAACTCAAGCTGACTGCCACTATTGGCGCCCTTAATACCCAACGCAATCGTACCAACCACTGCCAGCCCTAAGGCAAATGCATGGGTTATCGCACTAAACATTTCATTATTAAATTCATAATGCTTTGATTTTGCCACGCGCATCTTCTACCTCCTTGGAGATCTCTAAAGCTGACGGGGTAAACTATATAAAATCCAAATAAATTTCTAAAAATAAAAAAGTCTGTCGATGAACAGACTTTTTTATTATAGTTTAAAGCAAACTTTTAAATATAATAAAAAGAGTTAGTTGAAATTTTCTACTAACTCTTTTTTATTTTTAGTTTTTAACTGCAGAAGCAAATTCTTCTTTAGCAAAAGCTTCATCGATGATAGCTTTCAATTGAGCGTGTAACTTTCCAAATTTACAAAAGCGACTCATAGAATTATTTCCTCCCGTTAAATAATAGATAACTATTAAAAATAGACAATACTTGCTCATAAGTAACGGTACTTAAATTGTTTACTTTGGCGTGTTTCATTGCTTGATGAAACTGATTTTTAGTAAACAGTTGACGATATTCTCGATTGACCCATTTTGAAACAAAGTACGTATATAGCTTCCAATATTTATCTGGAACATCTGTGGTATGGCGGGTAAGTTTTATTAAGACACTGTTTACTTTTGGTTTAGGATGAAAGCATTCCGCTGGCAGCTTAAGCAATTGCTGAATCGAGACTTGAGTGTGCAAGAGCAACCCTAGTGTTCGGTGAATATCCAAGGTACGCTTGTAGAATCCTTCTTCAACAATCAGATAGATGTCAGACGCATGGCTTTCAAAAACCACTTTTTTAATAATTTGTGTGCTTAAATGGTAAGGAATACTCCCAACAATTTTATACCTCTGTTTGTTAGGGAATTGAAACTGTAGAATATCTTGGTGAATTAAAGTGACACGAGTATTCAGTTTTAATTTTTCTGACGATAAGTTGAATAGATGACTGTCTAATTCAATAGACGTTACCTGTTTACTTATTTTAGCCAGTTTCGTCGTTAAATGCCCTTTACCTGTTCCAATTTCGTAAACGGTATCGGTTTCTTTTAAATTCAATTGTTTTATTATTTGGTTGAGTACTTTTTCACTCGTTAAAAAGTTTTGAGAATATTTTATATTTTTGTTCATGTAATCACTCCTTCTTAATTACAAATTTTTAGCATCTAATTTAACTTCAATTCCTATTATACAAAATTTTAAGATACTGCACTATCAACACACTCTTAAGTTTGCTTCTAAGTCTTATTTCCATAACTTCTTTTACGTTTCCGCCATTCTTTGCTGTTTCGATTTTTATGATATGGTGCAAGTCAGCACGAACACGAACCGTCTTATCTCCCATTATATCTTTTTTTGCACTGATTGGTGTATCATTTCGTTTTTCTTTTGCGGACCTGCAGATGCGATATCATGCGCATGCAAGCTTATCGATGATAAGCTGTCAAACATGAGAATTACAACTTATATCGTATGGGGCTGACTTCAGGTGCTACATTTGAAGAGATAAATTGCACTGAAATCTAGAAATATTTTATCTGATTAATAAGATGATCTTCTTGAGATCGTTTTGGTCTGCGCGTAATCTCTTGCTCTGAAAACGAAAAAACCGCCTTGCAGGGCGGTTTTTCGAAGGTTCTCTGAGCTACCAACTCTTTGAACCGAGGTAACTGGCTTGGAGGAGCGCAGTCACCAAAACTTGTCCTTTCAGTTTAGCCTTAACCGGCGCATGACTTCAAGACTAACTCCTCTAAATCAATTACCAGTGGCTGCTGCCAGTGGTGCTTTTGCATGTCTTTCCGGGTTGGACTCAAGACGATAGTTACCGGATAAGGCGCAGCGGTCGGACTGAACGGGGGGTTCGTGCATACAGTCCAGCTTGGAGCGAACTGCCTACCCGGAACTGAGTGTCAGGCGTGGAATGAGACAAACGCGGCCATAACAGCGGAATGACACCGGTAAACCGAAAGGCAGGAACAGGAGAGCGCACGAGGGAGCCGCCAGGGGGAAACGCCTGGTATCTTTATAGTCCTGTCGGGTTTCGCCACCACTGATTTGAGCGTCAGATTTCGTGATGCTTGTCAGGGGGGCGGAGCCTATGGAAAAACGGCTTTGCCGCGGCCCTCTCACTTCCCTGTTAAGTATCTTCCTGGCATCTTCCAGGAAATCTCCGCCCCGTTCGTAAGCCATTTCCGCTCGCCGCAGTCGAACGACCGAGCGTAGCGAGTCAGTGAGCGAGGAAGCGGAATATATCCTGTATCACATATTCTGCTGACGCACCGGTGCAGCCTTTTTTCTCCTGCCACATGAAGCACTTCACTGACACCCTCATCAGTGCCAACATAGTAAGCCAGTATACACTCCGCTAGCGCTGATGTCCGGCGGTGCTTTTGCCGTTACGCACCACCCCGTCAGTAGCTGAACAGGAGGGACAGCGTGTTGCTTTGATTGATAGCCAAAAAGCAGCAGTTGATAAAGCAATTACTGATATTGCTGAAAAATTGTAATTTATAAATAAAAATCACCTTTTAGAGGTGGTTTTTTTATTTATAAATTATTCGTTTGATTTCGCTTTCGATAGAACAATCAAAGCGAGAATAAGGAAGATAAATCCCATAAGGGCGGGAGCAGAATGTCCGAGACTAATGTAAATTTGTCCACCAATTAAAGGACCGATAACGCGCTCGAGCTGATTATCTGGCTCAGCAACAAACCAAGCAGCCGGCAGCCAAACCAAAAACATCAGCCGCTGACGCTGTACCGAAAAAAGCCGCGCCAAAGAAGAAAACAAAACTGACATATGCTGAGCAGATAGAGTATGATAAACTCCAAAATGAGCTTGACGAGTTGGACGATAAACTCGCCAAAGTCAAGGCAGCTATGGCCGAAGTCAACGGTGAAGATTATGTCAAACTAGGTGACTTACAAGCCCAAATTGACAAGATCAACCAAACAATTGATAAAAAATTCGACCGATTTGCCGAACTGGATCAGTATGTTTGAACACACGCCCACTGGAGGGAAGAAGACAATGTTAGAGCAGCCATATCTCGATCTTGCCCAAAAAGTATTAGATGAAGGCCATTTCAAGCCTGATCGCACGCATACAGGCACGTACAGTATTTTTGGTCACCAAATGCGGTTTGACCTTAGCAAAGGGTTTCCTTTACTAACAACCAAAAAGGTGCGCTTTGGTCAAGCTTGATAAACAATTTAAATCTACCGTTCGTATAATGTATGCTATACGAAGTTATGACAATGTCTTAGGCGTTAAGGTCGTTTTAGCCGATGGTCGCGAAGTTAAGTAAGGTACCATGCAGTTTAAATTCGGTCCTCGGGATATGATAAGATTAATAGTTTTAGCTATTAATCTTTTTTTATTTTTATTTAAGAATGGCTTAATAAAGCGGTTACTTTGGATTTTTGTGAGCTTGGACTAGAAAAAAACTTCACAAAATGCTATACTAGGTAGGTAAAAAAATATTCGGAGGAATTTTGAAATGGCAATCGTTTCAGCAGAATGCAGATGAAGAAAGCAGACAAGTAAGCCTCCTAAATTCACTTTAGATAAAAATTTAGGAGGCATATCA

SEQ ID NO: 4 - protein, 21 aa, Lactobacillus casei
TCGGCTAAAACGACCTTAACG

SEQ ID NO: 5 - protein, 18 aa, Lactobacillus casei
TAGGGACCCATAACTTCG

SEQ ID NO: 6 - protein, 21 aa, Lactobacillus casei
ATATCAAAAAAAAGCCCGCTC

SEQ ID NO: 7 - protein, 28 aa, Lactobacillus casei
TCAGATCTAGTCTTATAACTATACTGAC

SEQ ID NO: 8 - protein, 26 aa, Lactobacillus casei
ATCTCGAGACTCGCATTGGGATTACC

SEQ ID NO: 9 - protein, 22 aa, Lactobacillus casei
TAAAGAAATCTGTACCGGTTGC

(19) United States
(12) Patent Application Publication
(10) Pub. No.: US 2018/0355023 A1
(43) Pub. Date: Dec. 13, 2018

(54) TREATMENT OF DISEASE WITH LACTIC ACID BACTERIA HAVING STABLY INTEGRATED TRAPPIN-2

(71) Applicant: Vithera Pharmaceuticals, Inc., Cambridge, MA (US)

(72) Inventors: Bernard Malfroy-Camine, Arlington, MA (US); Laura Holberger, Cambridge, MA (US); Johannes Fruehauf, Newton, MA (US)

(73) Assignee: Vithera Pharmaceuticals, Inc., Cambridge, MA (US)

(21) Appl. No.: 15/761,277

(22) PCT Filed: Sep. 21, 2016
(86) PCT No.: PCT/US2016/052761

§ 371 Date: Mar. 19, 2018

Related U.S. Application Data
(60) Provisional application No. 62/284,140, filed on Sep. 21, 2015.

(51) Int. Cl. C07K 14/81; A61K 35/74; A61K 38/55

(52) U.S. Cl. CPC C07K 14/811; A61K 38/55; A61K 35/74

(57) ABSTRACT

The instant invention comprises Trappin-2 expressed through stable integration into the genome of a lactic acid bacteria useful in the treatment of diseases characterized by damaging elastolytic activity, or bacterial infection.

### FIELD OF THE INVENTION

The instant invention relates to Trappin-2, or pre-elafin, its manufacture, and its therapeutic use. Trappin-2 is a 95 amino acid polypeptide comprised of two domains, a WAP domain and a Cementoin domain. Its WAP (whey acidic protein) domain, sometimes referred to as ‘elafin’, confers it a potent anti-elastase activity, while its Cementoin domain confers it a broad antibacterial activity. Trappin-2 has therapeutic potential as an anti-inflammatory drug inhibiting the proinflammatory enzyme elastase, and also as an antimicrobial drug.

### BACKGROUND OF THE INVENTION

The potential of trappin-2 as an anti-inflammatory drug has been established in mouse models for Inflammatory Bowel disease (IBD). IBD comprises pathologies such as ulcerative colitis or Crohn's disease, which are characterized by severe inflammation of the colon. It has been shown that the epithelial lining of the colon in IBD exhibits dramatically increased elastolytic activity, suggesting that this activity might be a possible target for treatment. Motta et al. (2011) have demonstrated that increased levels of Trappin-2 (elafin) achieved either in transgenic mice overexpressing elafin or through intracolonic administration of adenoviral vectors expressing elafin, were protected against trinitrobenzene sulfonic acid (TNBS) or dextran sodium sulfate (DSS)-induced colitis, a well-recognized mouse model for IBD. In a subsequent work the same authors demonstrated that a food grade bacteria such as Lactococcus lactis (L-lactis) or Lactobacillus casei (L-casei) transfected with a plasmid encoding trappin-2 (elafin) administered orally could also protect mice from TNBS- or DSS-induced colitis (Motta et al., 2012). Without being bound to any particular theory, this data further substantiated the potential of trappin-2 as a treatment for IBD in humans and suggested that it should be possible to design a therapeutic treatment utilizing a food grade bacteria such as L-casei or L-lactis as a delivery system of trappin-2 to the gut.

However it would be very difficult if not impossible to develop such a treatment for human use if trappin-2 expression was achieved through transfection with a plasmid, as large scale production following cGMP guidelines of pharmaceutical grade transfected bacteria would be extremely difficult to achieve consistently and even if possible would likely be prohibitively expensive and not commercially viable. However stable integration of the trappin-2 gene in the genome of L-casei or L-lactis would yield a recombinant bacteria that would be much easier to produce in large amounts under cGMP conditions.

Therefore there is a need for a method to produce a recombinant food grade bacteria such as L-lactis or L-casei having the trappin-2 stably integrated in its genome and capable of secreting trappin-2. Trappin-2; however, has broad antibacterial activity through its Cementoin domain, creating potentially unsurmountable difficulty in engineering a viable bacteria capable of directly secreting trappin-2. Indeed, while conceivably transfection of a large amount of healthy bacteria with a plasmid encoding trappin-2 could be expected to yield some degree of trappin-2 secretion, it is completely counter-intuitive to expect a bacteria to secrete an antibacterial polypeptide as the polypeptide would be expected to be cytotoxic. This difficulty has long been recognized. For example Ishima et al. (U.S. Pat. No. 5,734,014) teach that in order to produce active recombinant elafin it was necessary to express elafin through a fusion protein in E-coli while direct expression was possible in yeast. More generally, the inherent difficulty of expressing antibacterial polypeptides in bacteria has been the subject of a number of publications describing specific strategies to circumvent the problem (see for example Skozyrev et al., 2003 for sarcotoxin IA, Barrel et al., 2004 for mangainins; Wei et al., 2005 for small antimicrobial peptides; Meiyalaghan et al., 2014 for Snakin peptides; Zorko et al. 2010 for a general strategy).

### BRIEF SUMMARY OF THE INVENTION

We have made the unexpected discovery that L-casei with the trappin-2 stably integrated in its genome is viable and directly secretes trappin-2, without any need for a fusion protein.

Because trappin-2 has a dual antibacterial and anti-elastase activity, a lactic acid bacteria having the trappin-2 stably integrated into its genome (subsequently referred to as a LAB-trappin-2) has great therapeutic potential in a number of diseases. They include diseases where elastase activity is increased and leads to tissue damage. For example, Motta reported an increased elastolytic activity in the epithelium of the gut in biopsies of patients suffering from ulceritis colitis. Therefore ulceritis colitis and more generally IBD may be treated with a LAB-trappin-2. Certain dermatitides, for example noninfectious granulomatous dermatitides, more specifically annular elastolytic giant cell granuloma, exhibit increased elastolytic activity (Goldminz et al. 2013). These may be treated with a topical formulation of a LAB-trappin-2.

Shrivastava et al. 2013 show that a mouth rinse with inhibitors of proteases (matrix metalloproteases) shows beneficial effects in radiation-induced mucositis. Elastolytic activity is increased in radiation-induced mucositis, which suggests that this pathology may benefit from a LAB-trappin-2 administered topically, for example by mouth rinsing.

Trappin-2 has broad antimicrobial activity against pathogenic bacteria including Pseudomonas aeruginosa (P. aeruginosa) and Staphylococcus aureus (S. aureus), two bacteria which are particularly difficult to eliminate. Baranger et al. (2008) tested the antibacterial properties of trappin-2 towards other pathogens. They found that trappin-2, at concentrations of 5-20 micromolar, has significant activity against Klebsiella pneumoniae, Haemophilus influenzae, Streptococcus pneumoniae, Branhamella catarrhalis and the pathogenic fungi Aspergillus fumigatus and Candida albicans, in addition to P. aeruginosa and S. aureus.

Thus a number of pathologies due to bacterial or fungal infections may benefit from suitable formulations of a LAB-trappin-2. For example an oral formulation may be used to treat bacterial necrotizing enterocolitis, a severe pathology that is often linked to P. aeruginosa (Leigh et al. 1995). As another example, an intra vaginal formulation may be used to treat bacterial vaginitis which often are due to S. aureus (Mumyaz et al., 2008). As yet another example, a topical formulation may be used to treat various skin bacterial and fungal infections. In these indications, a LAB-trappin-2 treatment would have distinct advantage over a simple LAB treatment.

The present invention according to the disclosure provides a food grade bacteria (L-lactis or L-casei) having the trappin-2 gene directly and stably integrated in its genome and suitable for pharmaceutical development. The present invention also provides methods of treatment of inflammatory diseases such as IBD, of dermatological conditions such as dermatitides, and of bacterial infections such as bacterial vaginitis, including vaginal pseudomonas infections, as well as gastrointestinal infections such as necrotizing enterocolitis.

In one illustrative embodiment according to the disclosure a pharmaceutical formulation of the lactic acid bacteria of invention the lactic acid bacteria expresses greater than about 10% the trappin-2 expressed by a second lactic acid bacteria containing the trappin-2 gene in the form of a plasmid.

In a further illustrative embodiment according to the disclosure the pharmaceutical formulation according to the disclosure the lactic acid bacteria is in the form of lyophilized probiotic pellets, wherein said lyophilized probiotic pellets are reduced to the desired particle size prior, which is about 60 to 800 microns.

In another illustrative embodiment the pharmaceutical formulation is encapsulated in a seamless soft gelatin capsule.

In yet a further illustrative embodiment according to the disclosure the pharmaceutical is in the form of an oral formulation selected from the group consisting of a milk drink, a yogurt-similar milk product, a cheese, an ice-cream, a fermented cereal-based product, a milk-based powder, an infant formula, a tablet, a capsule, a liquid suspension, a dried oral grit, a powder, a wet oral paste or jelly and a fluid.

In a further illustrative embodiment according to the disclosure the pharmaceutical formulation contains viable bacteria in each dosage form may be in the range of about 104-106 or greater, or in the range of 105 to 106 per unit dosage form.

In another illustrative embodiment according to the disclosure the pharmaceutical formulation contains viable bacteria in each dosage form will be about 1.0 to 10000 mg, wherein said viable bacteria in each capsule will be about 100 to about 5000 mg of probiotic bacteria.

In a further illustrative embodiment according to the disclosure a method of treating a human having a disease taken from the group consisting of IBD and IBS comprises the use of a pharmaceutical formulation of a first trappin-2 expressing lactic acid bacteria expressing greater than about 10% of a second lactic acid bacteria containing the trappin-2 gene in the form of a plasmid, wherein the survival of said first trappin-2 expressing lactic acid bacteria is greater than 10% of the potential finished product of a preparation of a third lactic acid bacteria not expressing trappin-2.

In yet another illustrative embodiment according to the disclosure the lactic acid bacteria comprises a recombinant gene coding for the elafin protein or an active fraction of the elafin protein, and selected from Lactococcus lactis or Lactobacillus casei.

In another illustrative embodiment according to the disclosure the lactic acid bacteria comprises a defective auxotrophic gene, whereby survival of said Lactic Acid Bacteria is strictly dependent upon the presence of specific compounds, wherein the defective auxotrophic gene is the thyA.

In a further illustrative embodiment according to the disclosure the Lactic Acid Bacteria is Lactococcus lactis inactivated in htrA gene.

### BRIEF SUMMARY OF THE DRAWINGS

FIG. 1 is a depiction of the Trappin-2 protein.

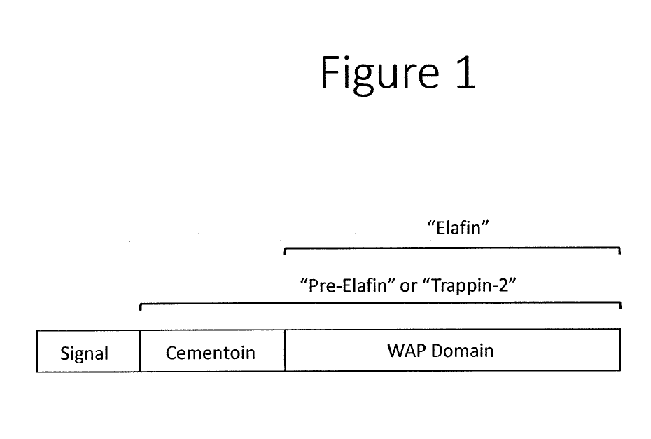

FIG. 2 is a depiction of the strategy used to stably integrate and express Trappin-2 from the chromosome of Lb. casei BL23.

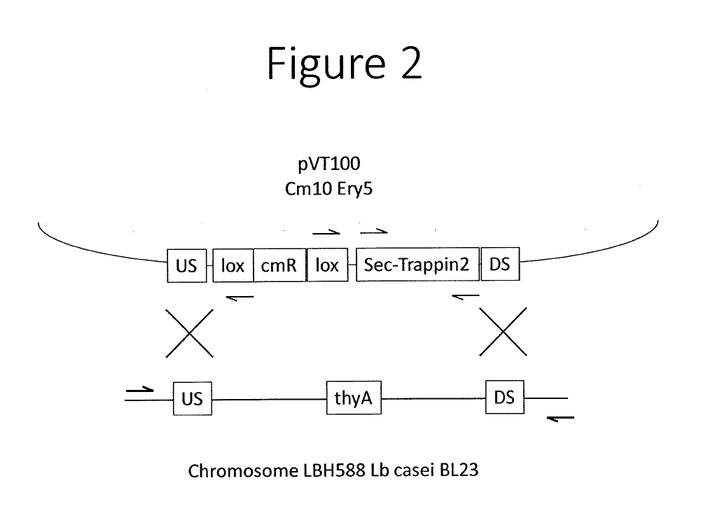

FIG. 3 shows the Trappin-2 sequence inserted into the thyA locus of Lb. casei BL23.

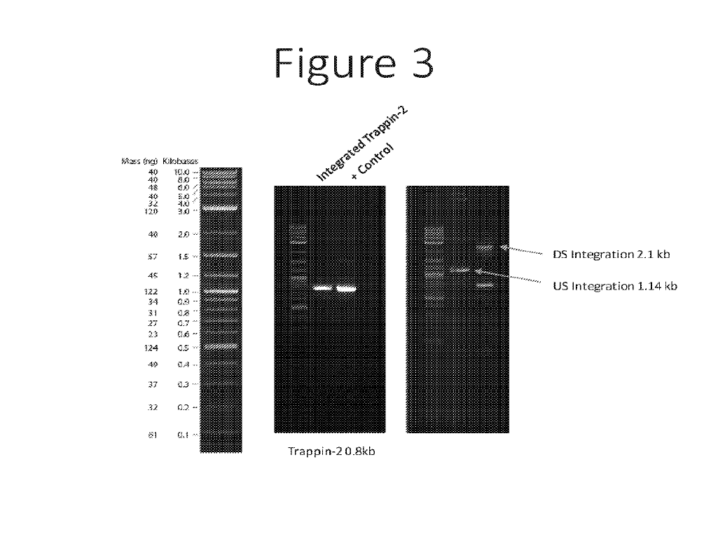

FIG. 4 shows Tappin-2 protein expression from the integrated thyA locus of Lb. casei BL23 as compared to expression from a transiently transfected plasmid.

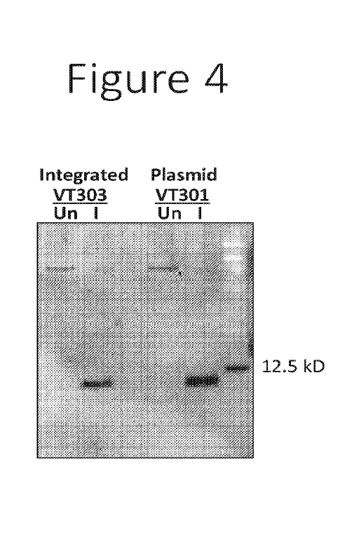

FIG. 5 shows the plasmid map for pVT100.

### DETAILED DESCRIPTION OF THE INVENTION

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each embodiment described herein is to be applied mutatis mutandis to each and every other embodiment unless specifically stated otherwise. Those skilled in the art will appreciate that the instant invention is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features. The instant invention is not to be limited in scope by the specific embodiments described herein, which are intended for the purpose of exemplification only. Functionally equivalent products, compositions and methods are clearly within the scope of the disclosure.

The instant invention is performed using conventional techniques of molecular biology, microbiology, virology, recombinant DNA technology, peptide synthesis in solution, solid phase peptide synthesis, and immunology. Such procedures are described, for example, in Sambrook, Fritsch & Maniatis, Molecular Cloning: A Laboratory Manual, told Spring Harbor Laboratories, New York, Second Edition (1989), whole of Vols I, II, and DI; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait; pp 1-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series; J. F. Ramalho Ortigao, “The Chemistry of Peptide Synthesis” In: Knowledge database of Access to Virtual Laboratory website (Interactiva, Germany); Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Wunsch, E., ed. (1974) Synthese von Peptiden in Houben-Weyls Metoden der Organischen Chemie (Muler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000), ISBN 0199637970, whole of text.

Probiotics

Probiotics are microbial-based dietary adjuvants that beneficially affect the host physiology by modulating mucosal and systemic immunity, as well as improving intestinal function and microbial balance in the intestinal tract (Naidu, A. S., et al. (1999), Probiotic spectra of lactic acid bacteria (LAB). Crit. Rev. Food Sci. Nutr. 39:3-126). Various nutritional and therapeutic effects have been ascribed to these probiotics including: modulating immune response, lowering serum cholesterol concentrations, improving lactose intolerance symptoms, increasing resistance to infectious intestinal diseases, decreasing diarrhea duration, reducing blood pressure, and helping to prevent colon cancer.

However, in order to exert these beneficial effects on the host, probiotics must retain their viability and reach the large intestine in large quantities (Favaro-Trindade, C. S., et al. (2002), J Microencapsulation 19(4): 485-494)). Effective probiotic bacteria should be able to survive gastric conditions and colonize the intestine, at least temporarily, by adhering to the intestinal epithelia (Conway, P. (1996), Selection criteria for probiotic microorganisms. Asia Pacific J. Clin. Nutr 5: 10-14).

As used herein, the term “probiotic” will be taken to mean a live microorganism which when administered in adequate therapeutic amounts confer a health benefit on a subject. Health benefits are a result of production of nutrients and/or co-factors by the probiotic, competition of the probiotic with pathogens and/or stimulation of an immune response in the subject by the probiotic. Exemplary probiotics are generally recognized as safe (GRAS).

As used herein, the term “generally recognized as safe” or “GRAS” refers to prokaryotic or eukaryotic microorganisms that, based on experimental data and practical use experience, have been found not to produce substantial levels of toxic or otherwise hazardous substances or to have adverse effects when ingested by higher organisms including humans and other mammals. A listing of exemplary microorganisms generally recognized as safe is available in the GRAS Notice Inventory at the US Food and Drug Administration (FDA). The group of GRAS organisms includes microorganisms that are conventionally used in the manufacturing of food products. Typical examples of such organism are the group of lactic acid bacteria that are used as starter cultures in the dairy industry, the feed industry and other industries concerned with the manufacturing of product where lactic acid bacterial cultures are used. This term also encompasses obligate anaerobic bacteria belonging to the Bifidobacterium genus which are taxonomically different from the group of lactic acid bacteria. Other examples of GRAS organisms are yeast species used in food manufacturing such as baker's yeast, brewer's yeast and yeast organisms used in the fermentation of wine and other beverages. Typical examples of yeast species that can be considered as GRAS organisms include Saccharomyces cerevisiae and Schizosaccharomyces pombe. The use of filamentous fungi having GRAS status is also contemplated within the scope of the disclosure.

In an illustrative embodiment of the instant disclosure, GRAS organism is stably integrated with genetic material encoding for a polypeptide, wherein said polypeptide is taken from the group consisting of elafin, trappin-2 and cementoin. In one illustrative embodiment of the instant invention said polypeptide is trappin-2. In a further illustrative embodiment of the instant invention, said GRAS organism is a probiotic. In a further illustrative embodiment of the instant invention, said probiotic is taken from the group consisting of lactococcus lactis and lactobacillus casei.

In one illustrative embodiment, the instant invention relates to a process of manufacturing a softgel capsule containing microencapsulated probiotic bacteria and to the product made according to this process. More specifically, the product of the invention is stable at room temperature for at least about 24 months.

Probiotics are microbial-based dietary adjuvants that beneficially affect the host physiology by modulating mucosal and systemic immunity, as well as improving intestinal function and microbial balance in the intestinal tract (Naidu, A. S., et al. (1999), Probiotic spectra of lactic acid bacteria (LAB). Crit. Rev. Food Sci. Nutr. 39:3-126). Various nutritional and therapeutic effects have been ascribed to these probiotics including but not limited to the following: modulating immune response, lowering serum cholesterol concentrations, improving lactose intolerance symptoms, increasing resistance to infectious intestinal diseases, decreasing diarrhea duration, reducing blood pressure, and helping to prevent colon cancer.

However, in order to exert these beneficial effects on the host, probiotics must retain their viability and reach the large intestine in therapeutic quantities (Favaro-Trindade, C. S., et al. (2002), J Microencapsulation 19(4): 485-494)). Effective probiotic bacteria should be able to survive gastric conditions and colonize the intestine, at least temporarily, by adhering to the intestinal epithelia (Conway, P. (1996), Selection criteria for probiotic microorganisms. Asia Pacific J. Clin. Nutr 5: 10-14).

Lactic acid bacteria or Lactobacilli are the most commonly used probiotic for incorporation into dairy products such as yogurts, fermented milks and kefirs, and their use is continually becoming more widespread. For example, they are now added in dietary supplement forms, such as powders, capsules and tablets. Bifidobacteria and Streptococci are also commonly used probiotic microorganisms. Lactic acid bacilli generally require an effective delivery system that retains probio-functional activities (i.e., gutadhesion/retention, production of bacteriocins/enzymes) after their revival (Salminen, S., et al. (1996), Clinical uses of probiotics for stabilizing the gut mucosal barrier: successful strains and future challenges. Antonie Van Leeuwenhoek 70:347-3581). Furthermore, in addition to increasing in vivo viability and gastrointestinal tract life span, prolonged shelf life at room temperature remains a challenge in the manufacture of effective commercial products. Though freeze-drying of the probiotic bacteria has been shown to be an effective process for preservation and delivery of probiotics, several physico-chemical factors such as humidity, aeration (oxygen availability), processing (i.e., agitation), and temperature could compromise the cell viability, shelf life and, accordingly its therapeutic use.

The stability, viability (i.e., viable microbial content) and quality of products containing probiotic bacteria have been problematic, as evidenced by scientific literature. In one study regarding yogurts, the experiments yielded evidence that 3 of 6 products tested contained no traces of live microorganisms and two contained only low concentrations. Shah (2000) Journal of Dairy Science, 83(4): 894-907. Similar reports have issued with regard to products containing probiotic bacteria distributed in solid dosage forms such as powders, capsules and tablets. The predominant challenges to stability of probiotic bacteria are water activity, physical stress of processing and temperature. It has also been challenging to apply protective measures, such as coatings, that will release the probiotic bacteria at the appropriate delivery site in the body and allow the probiotic to colonize. The appropriate delivery and colonization of the coated probiotic bacteria has recently been confirmed in a newly published study (Del Piano, M., et al. (2010, Evaluation of the intestinal colonization by microencapsulated probiotic bacteria in comparison to the same uncoated strains, Journal of Clinical Gastroenterology, 44 Supp. 1: S42-6.)

Oil suspensions have been utilized to increase the viability and shelf life of probiotics. For example, U.S. Patent Application Publication No. 2004/0223956 discloses a composition containing probiotic bacteria suspended in an edible oil and, optionally, encapsulated in a two piece hard shell capsule. In addition, those in the art have tried using probiotic microspheres to enhance viability and shelf life. For example, U.S. Patent Application Publication No. 2005/0266069 discloses probiotic formulations containing probiotic microspheres having a core of a probiotic bacteria and a cellulosic excipient coated with coating agents and plasticizers.

Examples of probiotic bacteria of the instant invention may be taken from the group consisting of strains of L. curvatus, L. casei, L. delbrueki, L. acidophilus, L. reuteri, L. plantarum, L. gasseri, L. lactis sp. Lactis, L. lactis sp. cremoris, L. heviticus, L. salivarius, L. brevis, S. thermophilics, B. breve, L. crispatus, S. Lactis, B. Dentium, B. Longum, B. bifidum, and B. infantis. Particular strains include L. acidophilus M252, MB425, M253, M254, MB358, MB359, MB422, MB423, MB424, MB442, MB443, ATCC4356 and DSM20052; L. reuteri DSM20016 and DSM20053; L. delbruekii, L. delbrueckii subsp. delbruekii DSM20074, DSM20076 and ATCC9469; L. curvatus MB67, MB68; L. casei MB459, ATCC1 1741, ATCC393, ATCC7469, DSM20011, DSM20024, and MB50; L. planterum MB396, ATCC8014, NCDO1 193; L. gasseri MB335; L. lactis sp. lactis MB445, DSM20481, MB447; L. lactis sp. cremoris DSM4645, DSM20069, MB446; 5. thermophilus MB418, MB417, MB419, MB420, MB421, MB426; B. breve MB202; L. crispatus ATCC33197; L. salivarius ATCC1 1741, ATCC1 1742; L. helveticus S36.2, S40.8; L. brevis ATCC4006, MB64, MB65; S. lactis MB405, MB406, MB407, MB408; B. dentium ATCC423; Bifido SP MB200; B. longum MB201; B. bifidium MB254, MB225; and B. infantis MB257.

In an illustrative embodiment of the instant invention, probiotic is stably integrated with genetic material encoding for a polypeptide, wherein said polypeptide is taken from the group consisting of elafin, trappin-2 and cementoin. In one illustrative embodiment of the instant invention said polypeptide is trappin-2. In a further illustrative embodiment of the instant invention, said probiotic is a lactic acid bacteria. In a further illustrative embodiment of the instant invention, said lactic acid bacteria is taken from the group consisting of strains Lcr35 and BL23.

In another illustrative embodiment of the instant invention, lactic acid bacteria is stably integrated with genetic material encoding for a polypeptide, wherein said polypeptide is taken from the group consisting of elafin, trappin-2 and cementoin. In one illustrative embodiment of the instant invention said polypeptide is trappin-2. In a further illustrative embodiment of the instant invention, said lactic acid bacteria is taken from the group consisting of lactococcus lactis and lactobacillus casei. In another illustrative embodiment of the instant invention, said lactic acid bacteria is taken from the group consisting of strains LCR35 and BL26.

Pharmaceutical Formulations

The term ‘pharmaceutical formulation’ as used in this disclosure of the instant invention shall have the meaning of a means of delivering a probiotic to a subject in need of receiving the probiotic. The various means of delivering a probiotic are described below.

Experience has long shown that pharmaceuticals or other items for human or animal consumption may be safely and conveniently packaged in a hard or soft gelatin shell (softgel). Filled one-piece soft capsules or softgels have been widely known and used for many years and for a variety of purposes and are capable of retaining a liquid fill material. Most frequently, softgels are used to enclose or contain consumable materials such as vitamins, minerals, fruits and botanical extracts and pharmaceuticals in a liquid vehicle or carrier.

Encapsulation within a soft capsule of a solution or dispersion of a nutritional or pharmaceutical agent in a liquid carrier offers many advantages over other dosage forms, such as compressed, coated or uncoated solid tablets, or bulk liquid preparations. Encapsulation of a solution or dispersion permits accurate delivery of a unit dose. Soft capsules provide a dosage form that is easy to swallow and need not be flavored, a good oxygen barrier (i.e., low oxygen permeability through the capsule shell), and tamper protection. Soft capsules are also more easily transported than food products and liquids, such as yogurt and milk.

Probiotics are commercially available in seamless or soft gelatin capsules. Bifa-15™ (Eden Foods, Inc., Clinton, Mich.) is a seamless microencapsulation delivery system for Bifidobacteria, claiming to contain three billion bacteria. The capsules are admixed with oligosaccharides, sweeteners and flavors and presented in individually wrapped, single dose aluminum tubes. The contents are poured into the mouth with the proviso that capsules be swallowed whole and not chewed. Ultra-Dophilus™ (Nature's Plus, Melville, N.Y.) is a conventional-sized soft gelatin capsule containing two billion viable freeze-dried L. acidophilus. Probiotocs12Plus™ are soft capsules containing 12 strains of lactic acid bacteria with the aim of a 900 colony forming units potency at the time of manufacture, and no refrigeration required.

In case the compositions of the instant invention are intended in the form of an oral formulation, they may be offered as a milk drink, a yogurt-similar milk product, a cheese, an ice-cream, a fermented cereal-based product, a milk-based powder, an infant formula, a tablet, a capsule, a liquid suspension, a dried oral grit or powder, a wet oral paste or jelly, a grit or powder for dry tube feeding or a fluid for wet tube feeding. Alternatively, the drink may be prepared before use from a dissolvable capsule containing the active ingredients. Preferably, the drink may be prepared before use by reconstituting a dry powder containing the lyophilized bacteria and the iron chelator or, alternatively, by reconstituting a dry powder containing the lyophilized bacteria with a physiological solution already comprising the chelator. The dry powder is preferably packaged into airtight and light-tight sachets, under air or nitrogen, under a noble gas or even under vacuum.

In an embodiment of the instant invention, probiotic encapsulated in a soft gel capsule is stably integrated with genetic material encoding for a polypeptide, wherein said polypeptide is taken from the group consisting of elafin, trappin-2 and cementoin.

Dosage

Apart from sensorial considerations, a dosage form must be sufficiently robust such that a sufficient number of viable probiotic bacteria survive manufacturing conditions and storage, in order to exert a beneficial effect when in use. This problem is compounded by the fact that it is particularly important to have a high viable microbial count in a unit dosage form intended to treat conditions in the oral cavity, because a high proportion of the probiotic bacteria can be expected to be lost to the oral cavity because of ingestion.

The count of viable probiotic bacteria obtained can be determined by standard laboratory dilution methods generally known in the art, such as plating a quantified dilution of bacteria onto Lactobacilli MRS agar plates (Difco n. 288130) containing 0.05% cysteine-HCl, incubation at 370 C for 48 hours in anaerobic cabinet (Forma Scientific, Mod 24), and then performing a colony count. Removal of the nutrient media may be conveniently carried out using Beckman centrifuge at 10,000 rpm and a temperature of 4 0 C. Pellets so formed may then be suspended in a sterile suspending fluid containing Skimmed milk (Difco) 5%, lactose 3%, Yeast extract (Difco) 0.5%, cysteine-HCl 0.02%, pH 7.0-7.2. The bacteria may be rapidly frozen at −80° C. and lyophilized in a known manner using, for example an Edwards Module YO Instrument.

The number of viable bacteria in each dosage form may be in the range of about 104-106 or greater, or in the range of 105 to 106 per unit dosage form. A typical dosage form will contain about 1.0 to 10000 mg, more particularly about 100 to about 5000 mg of probiotic bacteria.

In an embodiment of the instant invention, a probiotic stably is integrated with genetic material encoding for a polypeptide, wherein said polypeptide is taken from the group consisting of elafin, trappin-2 and cementoin, and wherein said probiotic is delivered to a subject in need at a dose of between about 1.0 to 10000 mg.

An illustrative embodiment of the instant invention is represented by the compositions intended for gastrointestinal use, to be administered as a drink, a capsule, an infant formula or even as a dairy product. In such a case, the selected bacterial strains may be used so that the amount of bacteria available to the individual corresponds to about 103 to about 1014 colony forming units (CFU) per day, from about 107 to about 1012 CFU per day, or from about 109 to about 1012 CFU per day.

In a further illustrative embodiment of the instant invention, probiotic stably integrated with genetic material encoding for a polypeptide, wherein said polypeptide is taken from the group consisting of elafin, trappin-2 and cementoin, and wherein said probiotic is delivered to a subject in need in the form of a drink wherein the dosage of said probiotic is about 100 to about 1050 CFU.

Manufacturing

Probiotics of the instant invention may be obtained from commercial sources, or they may be obtained from laboratory strains. Said probiotics can be grown to log phase in a nutrient media according to techniques known in the art. Suitable media include MRS lactobacilli agar (Difco), or any other enriched media suitable for the cultivation of such media. The probiotics can be recovered from the culture medium in the form of a pellet by using centrifuge and filtration techniques generally known in the art. The pellet of probiotics thus formed is thereafter dried by lyophilisation.

Lyophilised probiotic pellets may be reduced to the required particle size prior to formulation. Suitable size reduction techniques include grinding and sieving according a process generally known to those skilled in the art. In one embodiment of the instant invention the probiotic mass is employed with a particle size in the range of 60 to 800 microns. Considering that the probiotic bacteria are formed from highly hygroscopic lyophilized material, and considering also that microbial growth is triggered by the presence of humidity, in order to keep the probiotics in a stable and quiescent state they must be maintained in a dry state at all times in the manufacturing process.

In an illustrative embodiment of the instant invention, a Lactobacillus probiotic is cultivated anaerobically in Lactobacilli MRS broth (DIFCO) for 16 hours at 370 C. To obtain a microbial biomass, cells are cultivated in a fermenter for 24 hours at 370 C. The culture obtained is centrifuged at 6000 rpm for 30 minutes to produce a pellet containing the cells. The pellet is then suspended in a suspending fluid (10% skimmed milk, 0.5% lactose, 0.5% yeast extract), freeze dried and used for tablet preparation, after grinding and sieving through a suitable screen to obtain granulates of the desired particle size in the range of 60 to 800 microns.

In a further illustrative embodiment of the instant invention, a probiotic is manufactured by a process comprising stably integrating the genetic material encoding for a polypeptide, whereby said polypeptide is taken from the group consisting of elafin, trappin-2 and cementoin. In another illustrative embodiment of the instant invention, said probiotic of said manufacturing process expresses greater than about 10% of the trappin-2 expressed by a second probiotic containing the trappin-2 gene in the form of a plasmid.

In a further illustrative embodiment of the instant invention, lactic acid bacteria is manufactured by a process comprising stably integrating the genetic material encoding for a polypeptide, whereby said polypeptide is taken from the group consisting of elafin, trappin-2 and cementoin. In a further embodiment of the instant invention, said lactic acid bacteria of said manufacturing process expresses greater than about 10% of the trappin-2 expressed by a second lactic acid bacteria containing the trappin-2 gene in the form of a plasmid.

In another illustrative embodiment of the instant invention, an elafin, trappin-2 or cementoin expressing lactic acid bacteria taken from the group consisting of Lcr35 and BL23 is prepared by stably integrating genetic material substantively encoding for elafin, trappin-2 or cementoin. In one illustrative embodiment of the instant invention, said lactic acid bacteria is prepared by stably integrating genetic material substantively encoding for trappin-2, were substantively is defined as greater than about 75%. In a further illustrative embodiment of the instant invention, said method of making the trappin-2 expressing lactic acid bacteria sees the survival of said trappin-2 expressing lactic acid bacteria as being greater than about 25% of the potential finished product of a second preparation of lactic acid bacteria not expressing trappin-2. In a yet a further illustrative embodiment of the instant invention, said method of making the trappin-2 expressing lactic acid bacteria sees the survival of said trappin-2 expressing lactic acid bacteria as being greater than about 1-10% of the potential finished product of a second preparation of lactic acid bacteria not expressing trappin-2. In another illustrative embodiment of the instant invention, said method of making the trappin-2 expressing lactic acid bacteria sees the survival of said trappin-2 expressing lactic acid bacteria as being greater than about 10-20% of the potential finished product of a second preparation of lactic acid bacteria not expressing trappin-2. In a further illustrative embodiment of the instant invention, said method of making the trappin-2 expressing lactic acid bacteria sees the survival of said trappin-2 expressing lactic acid bacteria as being greater than about 20-30% of the potential finished product of a second preparation of lactic acid bacteria not expressing trappin-2.

The instant invention comprises an embodiment whereby a means for manufacturing trappin-2 sees at least about 80% of the trappin-2 gene being stably integrated into lactic acid bacteria. In a further embodiment of the instant invention, said means for manufacturing the lactic acid bacteria sees the survival of said trappin-2 expressing lactic acid bacteria being greater than about 25% of the potential finished product of a preparation of second lactic acid bacteria not expressing trappin-2.

The instant invention further comprises an embodiment whereby a means for manufacturing trappin-2 sees at least about 60-70% of the trappin-2 gene being stably integrated into lactic acid bacteria. In a further illustrative embodiment of the instant invention, said means for manufacturing the lactic acid bacteria sees the survival of said trappin-2 expressing lactic acid bacteria being greater than about 25% of the potential finished product of a preparation of second lactic acid bacteria not expressing trappin-2. The instant invention comprises an embodiment whereby a means for manufacturing trappin-2 sees at least about 70-80% of the trappin-2 gene being stably integrated into lactic acid bacteria. In a further illustrative embodiment of the instant invention, said means for manufacturing the lactic acid bacteria sees the survival of said trappin-2 expressing lactic acid bacteria being greater than about 25% of the potential finished product of a preparation of second lactic acid bacteria not expressing trappin-2.

The instant invention comprises an illustrative embodiment whereby a means for manufacturing trappin-2 sees at greater than about 80% of the trappin-2 gene being stably integrated into lactic acid bacteria. In a further embodiment of the instant invention, said means for manufacturing the lactic acid bacteria sees the survival of said trappin-2 expressing lactic acid bacteria being greater than about 25% of the potential finished product of a preparation of second lactic acid bacteria not expressing trappin-2.

The instant invention comprises an embodiment whereby a means for manufacturing trappin-2 sees at least about 80% of the trappin-2 gene being stably integrated into lactic acid bacteria. In a further illustrative embodiment of the instant invention, said means for manufacturing the lactic acid bacteria sees the survival of said trappin-2 expressing lactic acid bacteria being greater than about 1-10% of the potential finished product of a preparation of second lactic acid bacteria not expressing trappin-2.

The instant invention comprises an embodiment whereby a means for manufacturing trappin-2 sees at least about 80% of the trappin-2 gene being stably integrated into lactic acid bacteria. In a further illustrative embodiment of the instant invention, said means for manufacturing the lactic acid bacteria sees the survival of said trappin-2 expressing lactic acid bacteria being greater than about 10-20% of the potential finished product of a preparation of second lactic acid bacteria not expressing trappin-2. The instant invention comprises an illustrative embodiment whereby a means for manufacturing trappin-2 sees at least about 80% of the trappin-2 gene being stably integrated into lactic acid bacteria. In a further embodiment of the instant invention, said means for manufacturing the lactic acid bacteria sees the survival of said trappin-2 expressing lactic acid bacteria being greater than about 20-30% of the potential finished product of a preparation of second lactic acid bacteria not expressing trappin-2.

In an illustrative embodiment of the instant invention, a pharmaceutical formulation of lactic acid bacteria having a substantive amount of the genetic material encoding for trappin-2 stably integrated, whereby said lactic acid bacteria expresses greater than about 10% the trappin-2 expressed by a second lactic acid bacteria containing the trappin-2 gene in the form of a plasmid. In another illustrative embodiment of the instant invention an oral formulation of lactic acid bacteria having a substantive amount of the genetic material encoding for trappin-2 stably integrated, whereby said lactic acid bacteria expresses greater than about 10% the trappin-2 expressed by a second lactic acid bacteria containing the trappin-2 gene in the form of a plasmid. In yet a further illustrative embodiment of the instant invention an oral formulation of lactic acid bacteria having a substantive amount of the genetic material encoding for trappin-2 stably integrated, whereby said lactic acid bacteria expresses greater than about 10% the trappin-2 expressed by a second lactic acid bacteria containing the trappin-2 gene in the form of a plasmid is used as a method of treating a human having a disease taken from the group consisting of bacterial vaginitis, necrotizing enterocolitis, IBD and IBS, wherein dose of said trappin-2 expressing lactic acid bacteria is between about 1.0 to 10000 mg.

In a further illustrative embodiment of the instant invention, a method of treating a human having a disease taken from the group consisting of bacterial vaginitis, necrotizing enterocolitis, IBD and IBS comprises the use of said pharmaceutical formulation of a lactic acid bacteria comprising stably integrated genetic material encoding for at least about 80% of trappin-2.

In a another illustrative embodiment of the instant invention said method of treating a human having a disease taken from the group consisting of IBD and IBS, comprises the use of a pharmaceutical formulation of a first trappin-2 expressing lactic acid bacteria expressing greater than about 10% of a second lactic acid bacteria containing the trappin-2 gene in the form of a plasmid, whereby the survival of said first trappin-2 expressing lactic acid bacteria is greater than about 25% of the potential finished product of a preparation of a third lactic acid bacteria not expressing trappin-2.

### EXAMPLES

### Example 1 Integration of Tappin-2 into thyA Locus of Lb. casei BL23

Methods of gene replacement or deletion of the thymidine synthase gene are known to the person skilled in the art and can be achieved by double homologous recombination with a non-replicative integration vector based on the plasmid pLox71 or pNZ5319, SEQID2. This is demonstrated by (Lambert et al., 2007).

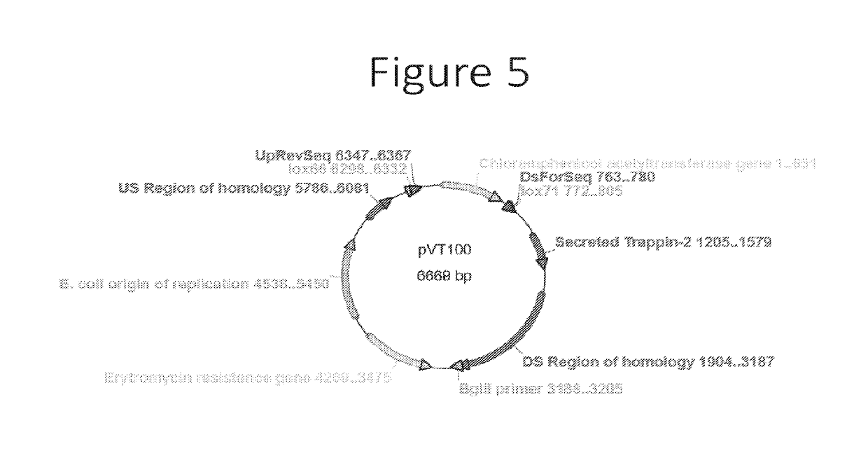

Plasmid pVT100, SEQID3, was constructed based on SEQID2 with regions of homology upstream and downstream of the thymidine synthase gene in Lactobacillus casei BL23. The gene for secreted Trappin-2 was sub-cloned upstream of the second region of homology (FIG. 5).

Transformation methods of Lactobacillus casei BL23 are known to the person skilled in the art and include, but are not limited to, protoplast transformation and electroporation. The expression plasmid which may contain part of the plasmid pVT100 can be transformed into recipient bacteria by these methods or others.

Trappin-2 integration was demonstrated by polymerase chain reaction (PCR) using primer sets consisting of one primer outside of the cloned regions of homology, UUS or DDS, SEQID 8 and 9 respectively, and one primer within the integration plasmid, UpRevSeq or DsRevSeq, SEQID 4 and 5. Integration events were indicated by PCR products containing both DNA sequences from the genome of Lactobacillus casei BL23 and the plasmid pVT100. Integration of the Trappin-2 gene was further verified using Trappin-2 forward and reverse primers, SEQID 6 and 7.

### Example 2

Expression of Tappin-2 Through thyA Locus of Lb. Casei BL23

Important to the function of this invention, Trappin-2 proteins are secreted from engineered lactic acid bacteria (LAB) from integrated DNA in the chromosome of Lactobacillus casei BL23. Trappin-2 proteins are present in supernatants from Trappin-2-expressing LAB and not in control supernatants when analyzed by SDS poly-acrylamide gel electrophoresis (SDS PAGE) and are reactive to anti-Elafin antisera by western blotting, a method used in the art as referenced in Current Protocols in Molecular Biology (Gallagher, 2006).

Trappin-2 expression was performed by growing a culture of LAB containing integrated Trappin-2 to logarithmic phase in MRS broth containing Chloramphenical at 37 degrees Celcius and then inducing Trappin-2 production. Trappin-2 was induced using 25 ng/mL nisin inducer for a period of 4 hours.

Trappin-2 expression was demonstrated by precipitating the protein from supernatants of induced and uninduced cultures of Trappin-2 integrated Lactobacillus casei BL23. Protein precipitates were reconstituted and separated by SDS PAGE before being western blotted with anti-elafin anti-sera.

The contents of any patents, patent applications, patent publications, or scientific articles referenced anywhere in this application are herein incorporated in their entirety.

SEQUENCES

SEQID1: Trappin-2
atgaaaaaaaagattatctcagctattttaatgtctacagtgatactttctgctgcagccccgttgtcaggtgtttatgcatcagcagctgtc
acgggagttcctgttaaaggtcaagacactgtcaaaggccgtgttccattcaatggacaagatcccgttaaaggacaagtttcagttaaa
ggtcaagataaagtcaaagcgcaagagccagtcaaaggtccagtctccactaagcctggctcctgccccattatcttgatccggtgcg
ccatgttgaatccccctaaccgctgcttgaaagatactgactgcccaggaatcaagaagtgctgtgaaggctcttgcgggatggcctgt
ttcgttccccagtga
SEQID2: pNZ5319
atgaactttaataaaattgatttagacaattggaagagaaaagagatatttaatcattatttgaaccaacaaacgacttttagtataaccaca
gaaattgatattagtgttttataccgaaacataaaacaagaaggatataaattttaccctgcatttattttcttagtgacaagggtgataaact
caaatacagcttttagaactggttacaatagcgacggagagttaggttattgggataagttagagccactttatacaatttttgatggtgtat
ctaaaacattctctggtatttggactcctgtaaagaatgacttcaaagagttttatgatttatacctttctgatgtagagaaatataatggttcg
gggaaattgtttcccaaaacacctatacctgaaaatgcttttctctttctattattccatggacttcatttactgggtttaacttaaatatcaata
ataatagtaattaccttctacccattattacagcaggaaaattcattaataaaggtaattcaatatatttaccgctatctttacaggtacatcatt
ctgtttgtgatggttatcatgcaggattgtttatgaactctattcaggaattgtcagataggcctaatgactggcttttataatatgagataatg
ccgactgtactttcggatcctaaacgcaattgatgattggttcggaaggcacgttaggaatcattaccgaagtaatcgttaaactgttgcc
gattccgctagggacccataacttcgtataatgtatgctatacgaacggtacagcccgggcatgagctccgatcgctacgagaagacg
cactatcgaccatacctaataatttatctacattccctttagtaacgtgaagaagatctctaaagctgacggggtaaactatataaaatcca
aataaatttctaaaaataaaaaagtctgtcgatgaacagacttttttattatagtttaaagcaaacttttaaatataataaaaagagttagttga
aattttctactaactcttttttatttttagtttttaactgcagaagcaaattcttctttagcaaaagcttcatcgatgatagctttcaattgagcgtg
taactttccaaatttacaaaagcgactcatagaattatttcctcccgttaaataatagataactattaaaaatagacaatacttgctcataagt
aacggtacttaaattgtttactttggcgtgtttcattgcttgatgaaactgatttttagtaaacagttgacgatattctcgattgacccattttga
aacaaagtacgtatatagcttccaatatttatctggaacatctgtggtatggcgggtaagttttattaagacactgtttacttttggtttaggat
gaaagcattccgctggcagcttaagcaattgctgaatcgagacttgagtgtgcaagagcaaccctagtgttcggtgaatatccaaggta
cgcttgtagaatccttcttcaacaatcagatagatgtcagacgcatggctttcaaaaaccacttttttaataatttgtgtgcttaaatggtaag
gaatactcccaacaattttatacctctgtttgttagggaattgaaactgtagaatatcttggtgaattaaagtgacacgagtattcagttttaat
ttttctgacgataagttgaatagatgactgtctaattcaatagacgttacctgtttacttattttagccagtttcgtcgttaaatgccctttacctg
ttccaatttcgtaaacggtatcggtttcttttaaattcaattgttttattatttggttgagtactttttcactcgttaaaaagttttgagaatattttata
tttttgttcatgtaatcactccttcttaattacaaatttttagcatctaatttaacttcaattcctattatacaaaattttaagatactgcactatcaa
cacactcttaagtttgcttctaagtcttatttccataacttcttttacgtttccgccattctttgctgtttcgatttttatgatatggtgcaagtcagc
acgaacacgaaccgtcttatctcccattatatctttttttgcactgattggtgtatcatttcgtttttcttttgcggacctgcagatgcgatatcat
gcgcatgcaagcttatcgatgataagctgtcaaacatgagaattacaacttatatcgtatggggctgacttcaggtgctacatttgaagag
ataaattgcactgaaatctagaaatattttatctgattaataagatgatcttcttgagatcgttttggtctgcgcgtaatctcttgctctgaaaa
cgaaaaaaccgccttgcagggcggtttttcgaaggttctctgagctaccaactctttgaaccgaggtaactggcttggaggagcgcagt
caccaaaacttgtcctttcagtttagccttaaccggcgcatgacttcaagactaactcctctaaatcaattaccagtggctgctgccagtg
gtgcttttgcatgtctttccgggttggactcaagacgatagttaccggataaggcgcagcggtcggactgaacggggggttcgtgcata
cagtccagcttggagcgaactgcctacccggaactgagtgtcaggcgtggaatgagacaaacgcggccataacagcggaatgaca
ccggtaaaccgaaaggcaggaacaggagagcgcacgagggagccgccagggggaaacgcctggtatctttatagtcctgtcgggt
ttcgccaccactgatttgagcgtcagatttcgtgatgcttgtcaggggggcggagcctatggaaaaacggctttgccgcggccctctca
cttccctgttaagtatcttcctggcatcttccaggaaatctccgccccgttcgtaagccatttccgctcgccgcagtcgaacgaccgagc
gtagcgagtcagtgagcgaggaagcggaatatatcctgtatcacatattctgctgacgcaccggtgcagccttttttctcctgccacatg
aagcacttcactgacaccctcatcagtgccaacatagtaagccagtatacactccgctagcgctgatgtccggcggtgcttttgccgtta
cgcaccaccccgtcagtagctgaacaggagggacagcgtgttgctttgattgatagccaaaaagcagcagttgataaagcaattactg
atattgctgaaaaattgtaatttataaataaaaatcaccttttagaggtggtttttttatttataaattattcgtttgatttcgctttcgatagaaca
atcaaagcgagaataaggaagataaatcccataagggcgggagcagaatgtccgagactaatgtaaatttgtccaccaattaaagga
ccgataacgcgctcgagcctgatagaaacagaagccactggagcacgtttaaacaatttaaatctaccgttcgtataatgtatgctatac
gaagttatgacaatgtcttaggcgttaaggtcgttttagccgatggtcgcgaagttaagtaaggtaccatgcagtttaaattcggtcctcg
ggatatgataagattaatagttttagctattaatctttttttatttttatttaagaatggcttaataaagcggttactttggatttttgtgagcttgga
ctagaaaaaaacttcacaaaatgctatactaggtaggtaaaaaaatattcggaggaattttgaaatggcaatcgtttcagcagaatgcag
atgaagaaagcagacaagtaagcctcctaaattcactttagataaaaatttaggaggcatatca
SEQID3: pVT100
atgaactttaataaaattgatttagacaattggaagagaaaagagatatttaatcattatttgaaccaacaaacgacttttagtataaccaca
gaaattgatattagtgttttataccgaaacataaaacaagaaggatataaattttaccctgcatttattttcttagtgacaagggtgataaact
caaatacagcttttagaactggttacaatagcgacggagagttaggttattgggataagttagagccactttatacaatttttgatggtgtat
ctaaaacattctctggtatttggactcctgtaaagaatgacttcaaagagttttatgatttatacctttctgatgtagagaaatataatggttcg
gggaaattgtttcccaaaacacctatacctgaaaatgctttttctctttctattattccatggacttcatttactgggtttaacttaaatatcaata
ataatagtaattaccttctacccattattacagcaggaaaattcattaataaaggtaattcaatatatttaccgctatctttacaggtacatcatt
ctgtttgtgatggttatcatgcaggattgtttatgaactctattcaggaattgtcagataggcctaatgactggcttttataatatgagataatg
ccgactgtactttcggatcctaaacgcaattgatgattggttcggaaggcacgttaggaatcattaccgaagtaatcgttaaactgttgcc
gattccgctagggacccataacttcgtataatgtatgctatacgaacggtacagcccgggcatgagctcgtttaagcttagtcttataact
atactgacaatagaaacattaacaaatctaaaacagtcttaattctatcttgagaaagtattggtaataatattattgtcgataacgcgagca
taataaacggctctgattaaattctgaagtttgttagatacaatgatttcgttcgaaggaactacaaaataaattataaggaggcactcaaa
atgagtacaaaagattttaacttggatttggtatctgtttcgaagaaagattcaggtgcatcaccacgcattacaagtatttcgctatgtaca
cccggttgtaaaacaggagactctgcatggatcccccgtctgaacgaacttaatgggaggaaaaattaaaaaagaacagttatgaaaa
aaaagattatctcagctattttaatgtctacagtgatactttctgctgcagccccgttgtcaggtgtttatgcatcagcagctgtcacgggag
ttcctgttaaaggtcaagacactgtcaaaggccgtgttccattcaatggacaagatcccgttaaaggacaagtttcagttaaaggtcaag
ataaagtcaaagcgcaagagccagtcaaaggtccagtctccactaagcctggctcctgccccattatcttgatccggtgcgccatgttg
aatccccctaaccgctgcttgaaagatactgactgcccaggaatcaagaagtgctgtgaaggctcttgcgggatggcctgtttcgttcc
ccagtgaggactagtgaattcgcggccgcctgcaggtcgacggtatcgatagcccgcctaatgagcgggcttttttttgatatcaagctt
atcgataccgtcgacctcgagtgcatattttcggcaatcttctcaatgagatgctcttcagcatgttcaatgatgtcgattttttattaaaacgt
ctcaaaatcgtttctgaaaacgaagcacatgcttgggctgccttgtcggcgagaaagcttattattggcgatatcgaagctgtttaagtga
cggttttgactgattgcagtaccatcagacgtatcaaaaacgagggggattttaaatggtagcatttttgtgggcgcaggatcgggatgg
tgtaatcggtaaagacggccatttgccatggcatttgccagatgatttgcattatttccggactcagactgaaggaaaaatgatggtggtt
gggcgtcgcacgtacgaaagttttccaaaacggccattaccagatcgtacgaacgtggttttgacgcaccaggctgattaccaggcac
caggcgcgattgtcttgcatcaggttgctgaagtgcttgattatgcgaaggaacatgcagatcaggcattagtcatcgccggtggtgct
caaatctttagcgcctttaaagacatggttgataccttgctcgtgacccgtctagctggcagttttgcaggtgacactaaaatgattccact
agattgggatgcgtttactaaaacctcaagccgaactgtcgaagatcaaaaccccgctttgacgcatacttatgaagtttggcaaaagc
aaaaatgatctgacgcgtttagcagctaaagaaattttttaaggaacttaaatagttatgtgcatttgtagttcgtttttttaactaaaattgact
catgtgcaaaaaagatcggcttctccgtctgacggagcgagccgatcttttttatatagttagcattaaacgaaaaggtaaattgaaatgt
acatgcacaggctgccgagaatgacaaacaggtgccagatgacatggatgtaggggatgcctttttgcaggtacagcatagcgccac
ctgtgtatgctaacccgccagcaaccagtaaccagaagccaatcggtcctaagtgatgccacagtggcaccatgccgattaagcaaa
gccagccgagaatgacgtaaatcatggtttccagatgcttgaagcgattcaagaagaacagcttgtagaggatgccgccaaagcaaa
gcgcccagatggcaattagcaagccgatccctaatggtccgccaatcgcgaccaaacagtaaggcaggtaggagccagcgattaat
atgaaaacgccagagtgatctaggacctgtaggacatggcgcgccttgctaaaatagaaaccgtggaacgccgttgaagccgtatat
aagatgatgagggaaatcccaaatcctaagtaactgattaactcaagctgactgccactattggcgcccttaatacccaacgcaatcgt
accaaccactgccagccctaaggcaaatgcatgggttatcgcactaaacatttcattattaaattcataatgctttgattttgccacgcgca
tcttctacctccttggagatctctaaagctgacggggtaaactatataaaatccaaataaatttctaaaaataaaaaagtctgtcgatgaac
agacttttttattatagtttaaagcaaacttttaaatataataaaaagagttagttgaaattttctactaactcttttttatttttagtttttaactgca
gaagcaaattcttctttagcaaaagcttcatcgatgatagctttcaattgagcgtgtaactttccaaatttacaaaagcgactcatagaatta
tttcctcccgttaaataatagataactattaaaaatagacaatacttgctcataagtaacggtacttaaattgtttactttggcgtgtttcattgc
ttgatgaaactgatttttagtaaacagttgacgatattctcgattgacccattttgaaacaaagtacgtatatagcttccaatatttatctggaa
catctgtggtatggcgggtaagttttattaagacactgtttacttttggtttaggatgaaagcattccgctggcagcttaagcaattgctgaa
tcgagacttgagtgtgcaagagcaaccctagtgttcggtgaatatccaaggtacgcttgtagaatccttcttcaacaatcagatagatgtc
agacgcatggctttcaaaaaccacttttttaataatttgtgtgcttaaatggtaaggaatactcccaacaattttatacctctgtttgttaggga
attgaaactgtagaatatcttggtgaattaaagtgacacgagtattcagttttaatttttctgacgataagttgaatagatgactgtctaattca
atagacgttacctgtttacttattttagccagtttcgtcgttaaatgccctttacctgttccaatttcgtaaacggtatcggtttcttttaaattcaa
ttgttttattatttggttgagtactttttcactcgttaaaaagttttgagaatattttatatttttgttcatgtaatcactccttataattacaaattttta
gcatctaatttaacttcaattcctattatacaaaattttaagatactgcactatcaacacactcttaagtttgcttctaagtcttatttccataactt
cttttacgtttccgccattctttgctgtttcgatttttatgatatggtgcaagtcagcacgaacacgaaccgtcttatctcccattatatcttttttt
gcactgattggtgtatcatttcgtttttcttttgcggacctgcagatgcgatatcatgcgcatgcaagcttatcgatgataagctgtcaaaca
tgagaattacaacttatatcgtatggggctgacttcaggtgctacatttgaagagataaattgcactgaaatctagaaatattttatctgatta
ataagatgatcttcttgagatcgttttggtctgcgcgtaatctcttgctctgaaaacgaaaaaaccgccttgcagggcggtttttcgaaggt
tctctgagctaccaactctttgaaccgaggtaactggcttggaggagcgcagtcaccaaaacttgtcctttcagtttagccttaaccggc
gcatgacttcaagactaactcctctaaatcaattaccagtggctgctgccagtggtgcttttgcatgtctttccgggttggactcaagacg
atagttaccggataaggcgcagcggtcggactgaacggggggttcgtgcatacagtccagcttggagcgaactgcctacccggaac
tgagtgtcaggcgtggaatgagacaaacgcggccataacagcggaatgacaccggtaaaccgaaaggcaggaacaggagagcg
cacgagggagccgccagggggaaacgcctggtatctttatagtcctgtcgggtttcgccaccactgatttgagcgtcagatttcgtgat
gcttgtcaggggggcggagcctatggaaaaacggctttgccgcggccctctcacttccctgttaagtatcttcctggcatcttccagga
aatctccgccccgttcgtaagccatttccgctcgccgcagtcgaacgaccgagcgtagcgagtcagtgagcgaggaagcggaatat
atcctgtatcacatattctgctgacgcaccggtgcagccttttttctcctgccacatgaagcacttcactgacaccctcatcagtgccaaca
tagtaagccagtatacactccgctagcgctgatgtceggcggtgcttttgccgttacgcaccaccccgtcagtagctgaacaggaggg
acagcgtgttgctttgattgatagccaaaaagcagcagttgataaagcaattactgatattgctgaaaaattgtaatttataaataaaaatc
accttttagaggtggtttttttatttataaattattcgtttgatttcgctttcgatagaacaatcaaagcgagaataaggaagataaatcccata
agggcgggagcagaatgtccgagactaatgtaaatttgtccaccaattaaaggaccgataacgcgctcgagctgattatctggctcag
caacaaaccaagcagccggcagccaaaccaaaaacatcagccgctgacgctgtaccgaaaaaagccgcgccaaagaagaaaac
aaaactgacatatgctgagcagatagagtatgataaactccaaaatgagcttgacgagttggacgataaactcgccaaagtcaaggca
gctatggccgaagtcaacggtgaagattatgtcaaactaggtgacttacaagcccaaattgacaagatcaaccaaacaattgataaaaa
attcgaccgatttgccgaactggatcagtatgtttgaacacacgcccactggagggaagaagacaatgttagagcagccatatctcgat
cttgcccaaaaagtattagatgaaggccatttcaagcctgatcgcacgcatacaggcacgtacagtatttttggtcaccaaatgcggttt
gaccttagcaaagggtttcctttactaacaaccaaaaaggtgcgctttggtcaagcttgataaacaatttaaatctaccgttcgtataatgt
atgctatacgaagttatgacaatgtcttaggcgttaaggtcgttttagccgatggtcgcgaagttaagtaaggtaccatgcagtttaaattc
ggtcctcgggatatgataagattaatagttttagctattaatctttttttatttttatttaagaatggcttaataaagcggttactttggatttttgtg
agcttggactagaaaaaaacttcacaaaatgctatactaggtaggtaaaaaaatattcggaggaattttgaaatggcaatcgtttcagca
gaatgcagatgaagaaagcagacaagtaagcctcctaaattcactttagataaaaatttaggaggcatatca
SEQID4: UpRevSeq
tcggctaaaacgaccttaacg
SEQID5: DsForSeq
tagggacccataacttcg
SEQID6: Trappin-2 Rev
atatcaaaaaaaagcccgctc
SEQID7: Trappin-2 For
tcagatctagtcttataactatactgac
SEQID8: UUS
ATCTCGAGACTCGCATTGGGATTACC
SEQID9: DDS
TAAAGAAATCTGTACCGGTTGC

### REFERENCES

The contents of the following references are incorporated in their entirety herein.
Baranger K, Zani M L, Chandenier J, Dallet-Choisy S and Moreau T. 2008. The antibacterial and antifungal properties of trappin-2 (pre-elafin) do not depend on its protease inhibitory function. FEBS L 275: 2008-2020
Barrell P J, Liew O W and Conner A J. 2004. Expressing an antibacterial protein in bacteria for raising antibodies. Protein Expr Purif 33: 153-159.
Goldminz A M and Gottlieb A B. 2013. Noninfectious Granulomatous Dermatitides: A Review of 8 Disorders (Part 2 of 3). Semin Cutan Med Surg 32: e1-e6.
Ishima Y, Okawa N, Yoshida M, Amagaya S, Kaji A. 1998. Elafin Derivatice. U.S. Pat. No. 5,734,014.
Leigh L, Stoll B J, Rahman M and McGowan J. 1995. Pseudomonas aeruginosa infection in very low birth weight infants: a case-control study. Pediatr Infect Dis J 14: 367-371.
Meiyalaghan S, Latimer J M, Kralick A V, Shaw M L, Lewis J G, Conner A J and Barrell P J. 2014. Expression and purification of the antibacterial peptide GSL1 in bacteria for raising antibodies. BMC research Notes 7: 777
Motta J-P, Magne L, Descamps D, Rolland C, Squarzoni-Dale C, Rousset P, Martin L, Cenac L, Balloy V, Huerre M, Frohlich L F, Jenne D, Wartelle J, Belaaouaj A, Mas E, Vinel J-P, Alric L, Chignard M, Vergnolle N and Sallenave J-M. 2011. Modifying the Protease, Antiprotease Pattern by Elafin Overexpression Protects Mice From Colitis. Gastroenterology 140: 1272-1282.
Motta J-P, Bermudez-Humaran L G, Deraison C, Martin L, Rolland C, Rousset P, Boue J, Dietrich G, Chapman K, Kharrat P, Vinel J-P, Alric L, Mas E, Sallenave J-M, Langella P and Vergnolle N. 2012. Food-Grade Bacteria Expressing Elafin Protect Against Inflammation and Restore Colon Homeostasis. Sci Transl Med 4: 158ra144.
Mumtaz S, Ahmad M, Aftab I, Akhtar N, Hassan M and Hamid A. 2008. Aerobic vaginal pathogens and their sensitivity pattern. J Ayub Med Coll Abbottabad 20: 113-117.
Shrivastava R and Deshmukh S. 2013. A Bew Therapeutic Approach to Treat Oral Mucositis Using Specific MMP Blockers in an Osmotically Active Solution. J Cancer Res and Treatment 1: 4-11.
Skozyrev V S, Kulesskiy E A, Yalchnin A V, Temirov Y V and Vinokurov L M. 2003. Expression of the recombinant antibacterial peptide sarcotoxin IA in Escherichia coli cells. Protein Expre Purif 28: 350-356.
Wei Q, Kim Y S, Seo J H, Jang W S, Lee I H and Cha H J. 2005. Facilitation of Expression and Purification of an Antimicrobial Peptide by Fusion with Baculoviral Polyhedrin in Escherichia coli. Appl Environ Microbiol 71: 5038-5043.
Zorko M and Jerala R. 2010. Production of recombinant antimicrobial peptides in bacteria. Methods Mol Biol 618: 61-76.

We claim:

1. A lactic acid bacteria comprising stably integrated genetic material encoding for a polypeptide, whereby said polypeptide is selected from the group consisting of elafin, trappin-2 and cementoin.

2. The lactic acid bacteria of claim 1, whereby said lactic acid bacteria is selected from the group consisting of lactococcus lactis and lactobacillus casei.

3. The lactic acid bacteria of claim 1, whereby said lactic acid bacteria is selected from the group consisting of strains Lcr35 and BL26.

4. A process of manufacturing lactic acid bacteria comprising stably integrating the genetic material encoding for a polypeptide, whereby said polypeptide is taken from the group consisting of elafin, trappin-2 and cementoin.

5. The process of claim 4, whereby said lactic acid bacteria expresses greater than about 10% of the trappin-2 expressed by a second lactic acid bacteria containing the trappin-2 gene in the form of a plasmid.

6. The process of claim 4, wherein said elafin, trappin-2 or cementoin expressing lactic acid bacteria is selected from the group consisting of Lcr35 and BL26.

7. The process of claim 4, wherein said elafin, trappin-2 or cementoin is prepared by stably integrating genetic material substantively encoding for elafin, trappin-2 or cementoin.

8. The process of making the trappin-2 expressing lactic acid bacteria of claim 6, whereby the survival of said trappin-2 expressing lactic acid bacteria is greater than about 10% of the potential finished product of a second preparation of lactic acid bacteria not expressing trappin-2.

9. A method for manufacturing trappin-2 whereby at least about 80% of the trappin-2 gene is stably integrated into lactic acid bacteria.

10. The method for manufacturing trappin-2 according to claim 9, wherein said means for manufacturing the lactic acid bacteria sees the survival of said trappin-2 expressing lactic acid bacteria being greater than about 10-20% of the potential finished product of a preparation of second lactic acid bacteria not expressing trappin-2.

11. The method for manufacturing trappin-2 according to claim 9 wherein said means for manufacturing trappin-2 sees at least about 80% of the trappin-2 gene being stably integrated into lactic acid bacteria.

12. The method for manufacturing the lactic acid bacteria of claim 9, whereby the survival of said trappin-2 expressing lactic acid bacteria is greater than about 10% of the potential finished product of a preparation of second lactic acid bacteria not expressing trappin-2.

13. The method for manufacturing the lactic acid bacteria of claim 9, said method for manufacturing the lactic acid bacteria sees the survival of said trappin-2 expressing lactic acid bacteria being greater than about 25% of the potential finished product of a preparation of second lactic acid bacteria not expressing trappin-2.

14. A method of treating a human having a disease taken from the group consisting of vaginal pseudomonas, necrotizing enterocolitis, IBD and IBS comprising the use of a pharmaceutical formulation of a lactic acid bacteria comprising stably integrated genetic material encoding for at least about 80% of trappin-2.
